(19) United States
(12) Patent Application Publication
(10) Pub. No.: US 2022/0027504 A1
(43) Pub. Date: Jan. 27, 2022

(54) METHOD, SYSTEM AND APPARATUS FOR SECURE COMMUNICATION OF COMMERCIAL &/OR CLINICAL INFORMATION WITH INTEGRITY OF DATA

(71) Applicant: Planet Intellectual Property Enterprises Pty Ltd, Box Hill (AU)

(72) Inventors: Eduardo VOM, Box Hill, Victoria (AU); Jeremy Phillip STIMSON, Fitzroy North, Victoria (AU); Andrew William Coventry O'HARE, Mitcham, Victoria (AU); Qerim Antonio SHAHINI, Box Hill, Victoria (AU)

(73) Assignee: Planet Intellectual Property Enterprises Pty Ltd, Box Hill (AU)

(21) Appl. No.: 17/296,053

(22) PCT Filed: Nov. 22, 2019
(86) PCT No.: PCT/AU2019/000147

§ 371 Date: May 21, 2021

(51) Int. Cl. G06F 21/62; G16H 10/65; G16H 40/67; H04L 9/08; H04L 29/08; H04L 12/46

(52) U.S. Cl. CPC G06F 21/6245; G16H 10/65; H04L 12/46; H04L 9/0838; H04L 67/12; G16H 40/67

(57) ABSTRACT

A system and method of communicating patient data over an interconnected computer data network, including loading instructions from a predetermined instruction set for the encryption and decryption of patient data onto a patient mobile communication device where the patient data to be encrypted for including in the secure data communication is exclusive of direct patient identifiers; loading instructions from the predetermined instruction set onto the patient mobile communication device for connecting to a transient data store which resides within the interconnected computer data network intermediate the patient mobile communication device and a clinic having a local data management hub; providing a secure communication of data that includes the encrypted patient data between the patient mobile communication device and the clinic wherein both the patient mobile communication device and the local data management hub of the clinic only transmit or receive data by subscription with the transient data store.

### RELATED APPLICATIONS

This application claims priority to Australian Provisional Patent Application No. 2018904471 in the name of Planet Intellectual Property Enterprises Pty Ltd, which was filed on 23 Nov. 2019, entitled “Method, System and Apparatus for Secure Communication of Commercial &/or Clinical Information with Integrity of Data” and the specification thereof is incorporated herein by reference in its entirety and for all purposes.

### FIELD OF INVENTION

The present invention relates to the secure storage and communication of personal data. It will be convenient to hereinafter describe the invention in relation to its broad application to the healthcare industry and it has been described in this context. However, it should be appreciated that the present invention is not limited to that use, only. For example, the present invention may find useful effect in any industry where a need exists to securely store and transmit personal data.

### BACKGROUND ART

Throughout this specification the use of the word “inventor” in singular form may be taken as reference to one (singular) inventor or more than one (plural) inventor of the present invention.

It is to be appreciated that any discussion of documents, devices, acts or knowledge in this specification is included to explain the context of the present invention. Further, the discussion throughout this specification comes about due to the realisation of the inventor and/or the identification of certain related art problems by the inventor. Moreover, any discussion of material such as documents, devices, acts or knowledge in this specification is included to explain the context of the invention in terms of the inventor's knowledge and experience and, accordingly, any such discussion should not be taken as an admission that any of the material forms part of the prior art base or the common general knowledge in the relevant art in Australia, or elsewhere, on or before the priority date of the disclosure and claims herein.

The inventor has recognised that within the field of ART (Assisted Reproductive Technology) there is a need for providing useful connectivity between clinical or medical instruments and their communication with EMRs (Electronic Medical Records). In the context of the description herein of the present invention, it is to be noted that use of the term “instrument(s)” is reference to medical devices used in the lab, which may need patient data to operate effectively. Furthermore, it has been recognised that whilst laboratory information may be integrated in some forms leading to a clinic or laboratory focus, there exists a lack of connectivity for patient data and the effective management of that patient data with the patient being mobile between the clinic, their home and elsewhere for that matter. By way of illustration, it is commonplace to have patients travel to a clinic and queue for measurements such as blood sampling etc that feeds into the decisions made by clinicians such as drug dosage for instance. For example, a patient utilising an ART clinic may need to arrive at a clinic early in the morning and wait for hours to have their blood sample taken and then return home where such a visit is required every alternate day. It may be more convenient to have the patient stay at home with a device(s) that can provide the relevant clinical information instead. However, a complex problem exists once this patient traveling problem is solved in terms of the complexity and regulation that needs to be addressed to have the patient's clinical data provided from home to the clinic. Therefore, there is a need for appropriate connectivity with the clinic to be established and maintained for patient data from the home or offsite from the clinic or laboratory.

Currently, healthcare clinics often operate with limited interconnectivity of data and instruments, and on a local network. This limits their ability to effectively manage data and provide other services such as home-based monitoring of patients. There is, of course, a need for the appropriate and proper handling of patient data to ensure effective security and privacy requirements are met. This contributes to a strong perception that clinics feel safer storing their patient data locally and avoiding external handling of such sensitive data. However, it is recognised that substantial efficiencies and improvement in healthcare would be realized by providing appropriate interconnectivity of data externally of the clinic.

A problem exists at present in providing a useful solution to the need for providing secure clinical data transmission external to a healthcare clinic that avoids persistent storage of data in an internetworked or ‘cloud based’ computer data network environment outside the healthcare clinic. In this respect, whilst existing cloud-based solutions may utilize encryption of transmitted patient data, the data is persistently stored in the cloud. This means that potentially sensitive data is stored for extended periods in the cloud with other data, where it may be the target of unauthorized copying, decryption, modification, tampering and/or deletion. FIG. 1 illustrates this traditional data transmission approach of the prior art using cloud storage of encrypted patient data.

Another existing cloud-based solution for data security is to completely de-identify the data before transmitting it to the cloud, by removing all direct and indirect patient identifiers. However, this renders the data of limited use to a clinic, as the data can no longer be correlated to the patient.

The processing and analysis of patient data over open or wide area computer data networks may introduce problems stemming from latency of transmitted data by virtue of its external storage.

Further to the above, each country has its own regulatory environment affecting the storage of all personal data in the cloud. Furthermore, the regulatory environment is also dynamic, making compliance challenging for custodians of personal data. One example of such custodians is a healthcare clinic.

Data from the ‘Internet of Things’ and home devices are currently not easily integrated with provider systems, such as those provided by healthcare clinics.

Generally speaking, cloud-based storage of object specific data such as patient data has the advantage of the data being able to be encrypted and made accessible to authorized users and systems. However, data may not be stored in one location and may be incomplete. Further, data may be more susceptible to unauthorized users and systems, which presents challenges for complying with requirements such as IT and legal requirements. This is not desirable to clinics, for example, from a security standpoint as patient data, patient account credentials and access permissions are all typically stored externally to the clinic.

A direct connection between a patient externally and a clinic would typically be enabled through use of a virtual private network (VPN) and this provides security risks. A VPN allows a remote computer access to a secure network, by essentially “extending” the secure network to include the remote computer to become a part of the VPN. Once that remote computer is on the network via the VPN, it poses a significant threat that must be managed by implementing security measures, such security group memberships. The remote computer becomes the responsibility of the business function which exists to secure the network, usually an IT department if one exists. A risk is that users can be inadvertently provided access to parts of the network which should be protected. Furthermore, the infrastructure that enables a remote computer connection to the network needs to be maintained. This is usually in addition to the infrastructure that exists for on-site access to the network by local computers. Accordingly, the VPN infrastructure and software must be kept up to date and stringent security policies applied.

Alternatively, healthcare Apps may be employed. In such Apps, a patient has direct access to their data. In these Apps, a patient consent model is also required to share data with third parties. Furthermore, it is typical to require the creation of a user account, the details of which are held either in the clinic or a cloud repository.

Specific examples of prior art techniques for handling clinical patient information follow.

U.S. Pat. No. 9,959,386 (Ohad et al, assigned to General Electric Company) discloses a cloud-based clinical information system and its method of use. It makes use of a hybrid cloud system, utilising a local edge device and remote cloud, in a clinical environment for managing the access of healthcare entities to healthcare information.

US patent publication 2016/0147952 (Garcia et al, assigned to General Electric Company) discloses a cloud-based clinical distribution system and its methods of use. In this disclosure, use is made of a hybrid cloud system in a clinical environment including apparatus comprising an edge device to mediate between a local information system associated with a local cloud system and a remote cloud system.

The following three prior art references are examples of systems in which patient information is exclusively stored in a cloud computing environment.

WO 2011/163017 (UNFV-FY, Inc.) discloses a method of delivering decision support systems and electronic health records for reproductive care, pre-conceptive care, fertility treatments, and other health conditions.

US 2014/0324457 (Kim et al) discloses an integrated computerized predicting system where a computerized patient system is connected through a web interface to a matching server, where a smart health care matching server is configured to receive a selection criteria from a patient at the patient computerized system. The matching server is configured to utilize the selection criteria, the EMRs, and the personal health records with a smart health care matching system application to predict an appropriate health care professional and/or insurance plan for the patient.

WO 2018/057801 (Beckton Dickinson and Company) discloses encryption systems and methods for medical devices. A medical device includes a connectivity module for establishing a communication channel with a cloud system. After obtaining a test result, the device can generate an unencrypted data block comprising a device identifier and an encrypted data block comprising a serial number of the device and the test result using an encryption key associated with the device identifier. The device can securely send the test result to the cloud system by transmitting the unencrypted data block and the encrypted data block to the cloud system via the communication channel.

US 2016/0139156 discloses apparatus, methods, and systems for home monitoring of physiological states and conditions. This disclosure is an example of a system where a patient is remotely monitored through a mobile application connected to a sensor device, with data stored in the cloud.

With the above discussion in mind it has been recognised that there can be two issues to address, namely, (1) with regard to data security, it isn't feasible to store clinical patient data in the cloud and, (2) with regard to identity, there is a need to keep a patient's identity from being linked between any of the communication infrastructure to the clinic and the patient's own mobile communication device that may be used to provide the connectivity between patient and clinic.

The preceding discussion of background art is intended to facilitate an understanding of the present invention only. The discussion is not an acknowledgement or admission that any of the material referred to is or was part of the common general knowledge as at the priority date of the application.

### SUMMARY OF INVENTION

It is an object of the embodiments described herein to overcome or alleviate at least one of the above noted drawbacks of related art systems or to at least provide a useful alternative to related art systems.

In a first aspect of embodiments described herein there is provided a method of communicating patient data over an interconnected computer data network between a patient having a patient mobile communication device and a clinic having a local data management hub which is operatively connected to the interconnected computer data network and comprises processor means adapted to operate in accordance with a predetermined instruction set, the method comprising the steps of:

loading instructions from the predetermined instruction set for the encryption and decryption of patient data onto the patient mobile communication device for secure data communication where the patient data to be encrypted for including in the secure data communication is exclusive of direct patient identifiers;

loading instructions from the predetermined instruction set onto the patient mobile communication device for connecting to a transient data store which resides within the interconnected computer data network intermediate the patient mobile communication device and the clinic;

providing a secure communication of data that includes the encrypted patient data between the patient mobile communication device and the clinic wherein both the patient mobile communication device and the local data management hub of the clinic only transmit or receive data by subscription with the transient data store.

The secure communication of data between the patient mobile communication device and the clinic may further include at least one linked patient identifier which, in combination with information stored only on the local data management hub, identifies a patient.

The steps of loading instructions from the predetermined instruction set onto the patient mobile communication device may be performed simultaneously by the patient mobile communication device scanning a machine-readable optical label that contains information comprising the respective instructions to be loaded.

The patient data may be stored only on one or a combination of either the local data management hub or the patient mobile device.

Patient data may be derived from instruments in communication with the patient mobile communication device.

The method may further include the step of:

linking patient data received at the local data management hub from the patient mobile communication device with a patient's EMR.

The instructions from the predetermined instruction set for the encryption and decryption of data may include an encryption key unique for the patient.

In another aspect of embodiments described herein there is provided a method of communicating client data over an interconnected computer data network between a client having a mobile communication device adapted for transmitting and receiving data over the network and an enterprise having a local data management hub which is operatively connected to the interconnected computer data network and comprises processor means adapted to operate in accordance with a predetermined instruction set, the method comprising the steps of:

loading instructions from the predetermined instruction set for the encryption and decryption of client data onto the mobile communication device for secure data communication where the client data to be encrypted for including in the secure data communication is exclusive of direct client identifiers;

loading instructions from the predetermined instruction set onto the mobile communication device for connecting to a transient data store which resides within the interconnected computer data network intermediate the user device and the local data management hub;

providing a secure communication of data that includes the encrypted client data between the mobile communication device and the enterprise wherein both the mobile communication device and the local data management hub of the clinic only transmit or receive data by subscription with the transient data store.

The transient data store may be dedicated to the clinic. Further, the transient data store may be a message queue with a defined time to live for queued messages of about 1 minute or less.

The interconnected computer data network may comprise one or a combination of:

an intranet;

a local area network;

a campus network;

a wide area network;

the internet.

In yet a further aspect of embodiments described herein there is provided a system for communicating patient data over an interconnected computer data network between a patient and a clinic, the system comprising:

a patient mobile communication device operably associated with the patient;

a local data management hub operable within the clinic and which is operatively connected to the interconnected computer data network and comprises processor means adapted to operate in accordance with a predetermined instruction set;

a transient data store which resides within the interconnected computer data network intermediate the patient mobile communication device and the clinic;

an application program adapted for being downloaded from the predetermined instruction set and residing on the patient mobile communication device and further adapted to;

load instructions from the predetermined instruction set onto the patient mobile communication device for the encryption and decryption of patient data for secure data communication where the patient data to be encrypted for including in the secure data communication is exclusive of direct patient identifiers; and

load instructions from the predetermined instruction set onto the patient mobile communication device for connecting to the transient data store;

wherein both the patient mobile communication device and the local data management hub of the clinic only transmit or receive data, that includes the encrypted patient data securely communicated between the patient mobile communication device and the clinic, by subscription with the transient data store.

The securely communicated data between the patient mobile communication device and the clinic may further include at least one linked patient identifier which, in combination with information stored only on the local data management hub, identifies a patient.

The local data management hub and the patient mobile communication device may include storage means respectively for storing patient data.

The system of preferred embodiments may further comprise medical instruments in communication with the patient mobile communication device for providing measurements from which the patient data is derived. The system may further include at least one patient EMR.

The processor means of the local data management hub operating in accordance with the predetermined instruction set may be adapted to link patient data received at the local data management hub from the patient mobile communication device with a patient's EMR.

The instructions from the predetermined instruction set for the encryption and decryption of data may include an encryption key unique for the patient.

In the preferred system, the transient data store is dedicated to the clinic, the transient data store is a message queue and the message queue has a defined time to live for queued messages of about 1 minute or less.

In the preferred system the interconnected computer data network comprises one or a combination of:

an intranet;

a local area network;

a campus network;

a wide area network;

the internet.

In still another aspect of embodiments the present invention provides a system for communicating client data over an interconnected computer data network between a client and an enterprise, the system comprising:

a mobile communication device operably associated with the client;

a local data management hub operable within the enterprise and which is operatively connected to the interconnected computer data network and comprises processor means adapted to operate in accordance with a predetermined instruction set;

a transient data store which resides within the interconnected computer data network intermediate the mobile communication device and the enterprise;

an application program adapted for being downloaded from the predetermined instruction set and residing on the mobile communication device and further adapted to;

load instructions from the predetermined instruction set onto the mobile communication device for the encryption and decryption of client data for secure data communication where the client data to be encrypted for including in the secure data communication is exclusive of direct client identifiers; and

load instructions from the predetermined instruction set onto the mobile communication device for connecting to the transient data store;

wherein both the mobile communication device and the local data management hub of the enterprise only transmit or receive data, that includes the encrypted client data securely communicated between the mobile communication device and the enterprise, by subscription with the transient data store.

In a preferred system the securely communicated data between the mobile communication device and the enterprise further includes at least one linked client identifier which, in combination with information stored only on the local data management hub, identifies a client.

In another aspect of preferred embodiments there is provided apparatus adapted to communicate patient data over an interconnected computer data network between a patient and a clinic, said apparatus comprising:

processor means adapted to operate in accordance with a predetermined instruction set,

said apparatus, in conjunction with said instruction set, being adapted to perform the method steps as disclosed herein.

In yet another aspect of preferred embodiments there is provided a computer program product comprising:

a computer usable medium having computer readable program code and computer readable system code embodied on said medium for communicating patient data between a patient and a clinic within a data processing system over an interconnected computer data network, said computer program product comprising:

computer readable code within said computer usable medium for performing the method steps as disclosed herein.

In yet still another aspect of embodiments of the present invention there is provided a method of uniquely associating a patient with a clinical record that is communicable to a clinic, the patient having a patient mobile communication device and the clinic having a local data management hub that comprises processor means adapted to operate in accordance with a predetermined instruction set and which is operatively connected to an interconnected computer data network, the method comprising the steps of:

downloading an application program from the local data management hub onto the patient mobile communication device;

generating, at the clinic, a unique ID that links only the unique ID to an EMR of the patient;

embedding the unique ID into the downloaded application program;

creating a unique PIN for the patient to access the application program;

operatively associating one or more medical instruments for use by the patient with the application program;

communicating patient data created by the patient's use of the one or more medical instruments to the application program where loaded instructions from the predetermined instruction set onto the mobile communication device encrypt the patient data for secure data communication over the interconnected computer data network via a transient data store to the clinic;

decrypting the patient data at the local data management hub;

using the unique ID to correlate the patient data with the patient at the local data management hub.

Embodiments of the present invention provide secure external data transmission by use of a linked patient identifier, cryptography, and a cloud-based message queue, which in turn provide security in that data is handled in a transitory manner rather than use of persistent storage of patient data in the cloud. Local storage of patient data is provided with the use of a hub device in a secure and on-premise environment rather than in the cloud.

Further embodiments of the present invention provide for synchronization of devices with EMRs by use of a hub device to securely link a mobile application to EMRs. In this respect, the mobile application does not contain any personal or patient identifiable information. This is enabled by an onboarding process.

Remote monitoring and other services may be provided by embodiments of the present invention with the hub device securely linked to a mobile application with paired devices. In this respect, devices can be paired with the mobile application and leverage secure data transmission with the hub and EMRs.

Other aspects and preferred forms are disclosed in the specification and/or defined in the appended claims, forming a part of the description of the invention.

In essence, embodiments of the present invention stem from the counterintuitive realization that the secure communication of object specific data, such as patient data, across wide area computer data networks including the cloud may be performed whilst maintaining the data's integrity by using local storage of the data as opposed to external cloud storage for instance, in conjunction with the use of message queuing to limit external handling of data to being only a transitory passage to avoid external storage and latency issues.

The prior art problems identified herein above have been addressed by the solution provided by embodiments of the present invention comprising a combination of a ‘linked patient identifier’ (para-identification information requiring central/hub) with cryptographic keys to transmit data securely via a cloud-based message queue configured to store data only until it is delivered to the intended receiver, for example a patient or clinic. At least one advantage of this is that security is provided for clinical data in a transitory fashion to avoid restrictions on data being handled outside the secure clinic environment.

The solution provided by embodiments of the invention also addresses the practicalities associated with managing various sources of data, including home and clinic-based devices, and electronic health records (EMRs).

Advantages provided by the present invention comprise the following:
Reliance on the cloud is limited, due to the use of a message queue;
Data is stored locally so that it can be analyzed and processed in real time (or near real time), whereas cloud-based storage can create latency issues;
Remote communication between the patient and the data stored locally in the clinic is achieved while avoiding a direct connection into the clinic;
The ability to easily and securely connect a range of devices and data sources, including wearables, mobile applications, RFID tags/systems, GPS tracking, workflow tools, lab equipment and electronic health records;
Patient data may be persistently stored within a clinic (in a hub device), rather than in the cloud. This means the data may be less susceptible to unauthorized access. The clinic also has greater control over the data, including the ability to mediate patient access to data;
By using a cloud-based message queue, externally transmitted data is only stored temporarily on the cloud (until it is delivered to the other party, or a time limit is reached), rather than in a persistent manner. This means the data may be less susceptible to unauthorized access;
By virtue of an onboarding process, a mobile application does not need the patient to create a user account. This avoids the entry and storage of personal information in the cloud, which is typically required by healthcare mobile applications;
The system of embodiments of the invention facilitates synchronization of home and local devices to a patient's EMRs in a secure manner.

From a patient point of view there is no longer a need to travel to a clinic to conduct routine measurements of clinical information. Furthermore, patient data may be safely and securely transmitted to a clinic from a remote location for a physician so that they may make decisions to be communicated back to the patient without a dependency on having patient information stored in a manner and at locations that present security, integrity and regulatory hurdles to having that patient information stored in the first place.

To reiterate some advantageous aspects of embodiments of the present invention a system and method of using a transit cloud (message queue) may be provided to push and pull information without the need to persistently store any data in the cloud or providing direct access to the provider's system where the information is stored (e.g. not requiring use of a virtual private network). Corruption protection may be provided for communicated data. The message queue can be dedicated by subscription to each of the patient mobile device and the clinic. Use of the system and method for external interactions with clients, for example to enable home-based devices to seamlessly and securely transfer information to a provider's system without persistent storage of any data in the cloud. Use of the system and method allows for connection with multiple data inputs such as sensors, devices, and schedules to a single mobile application that communicates with the provider's system. The provider's system is able to link the data sent by the mobile application to the patient's/client's files.

Further scope of applicability of embodiments of the present invention will become apparent from the detailed description given hereinafter. However, it should be understood that the detailed description and specific examples, while indicating preferred embodiments of the invention, are given by way of illustration only, since various changes and modifications within the spirit and scope of the disclosure herein will become apparent to those skilled in the art from this detailed description.

### BRIEF DESCRIPTION OF THE DRAWINGS

Further disclosure, objects, advantages and aspects of preferred and other embodiments of the present invention may be better understood by those skilled in the relevant art by reference to the following description of embodiments taken in conjunction with the accompanying drawings, which are given by way of illustration only, and thus are not limitative of the disclosure herein, and in which:

FIG. 1 illustrates a prior art system;

FIG. 2 is a system diagram illustrating a general infrastructure of a system in accordance with a preferred embodiment of the present invention;

FIG. 3 is a system diagram illustrating a more detailed infrastructure in accordance with a preferred embodiment of the present invention;

FIG. 4 is a flow diagram illustrating am onboarding process for a patient in accordance with an embodiment of the present invention involving a method of uniquely associating a patient with a clinical record;

FIGS. 5a to 5c are flow diagrams illustrating a method of uniquely associating a patient with a clinical record in accordance with further embodiments of the present invention.

FIG. 6 is a schematic view of an embodiment of the present invention in which a database containing linked patient identifier is correlated to EMR's of a patient.

FIG. 7 is a schematic view of a communication system between a clinic and home premises fora patient in accordance with a preferred embodiment of the present invention.

FIG. 8 is a schematic view of the structure of a message queue to facilitate communication between a clinic and home premises for a patient in accordance with a preferred embodiment of the present invention.

FIG. 9 schematically illustrates the connectivity between EMR's and an instrument in accordance with a preferred embodiment of the present invention.

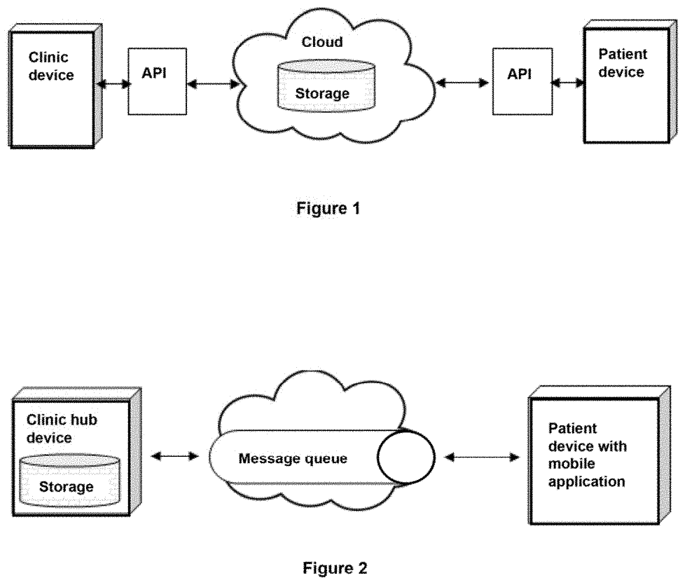

### DETAILED DESCRIPTION

In preferred embodiments of the present invention a solution is provided for the secure management and communication of patient data with a healthcare clinic, which involves transient use of networks to communicate personal data that avoids patient data being stored in the cloud for extended periods. Furthermore, in preferred forms the present invention provides middleware software to integrate all relevant healthcare instruments with EMR's to provide an enriched data source for clinical treatment.

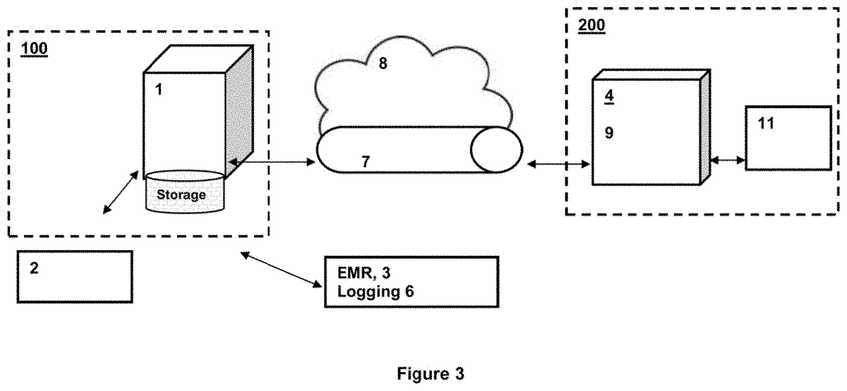

In the embodiment as illustrated in FIG. 3, a hub 1 (also referred to herein as the “Qbox” or “Qbox device”) is located within a clinic 100 and the hub device 1 functions as an integrated data management hub. The hub 1 provides single integration for multiple systems including local devices 2, EMRs 3, and home-based devices 4, with their own linked devices 11 in turn, which are associated with a patient. Middleware adapted for enabling connection between medical instruments, other devices and EMR's 3 resides on the hub 1. Advantageously, message queue connectivity credentials along with public encryption keys are generated at the hub 1 for use. Preferably, these are encoded within a QR code generated at the hub 1 for use in the system.

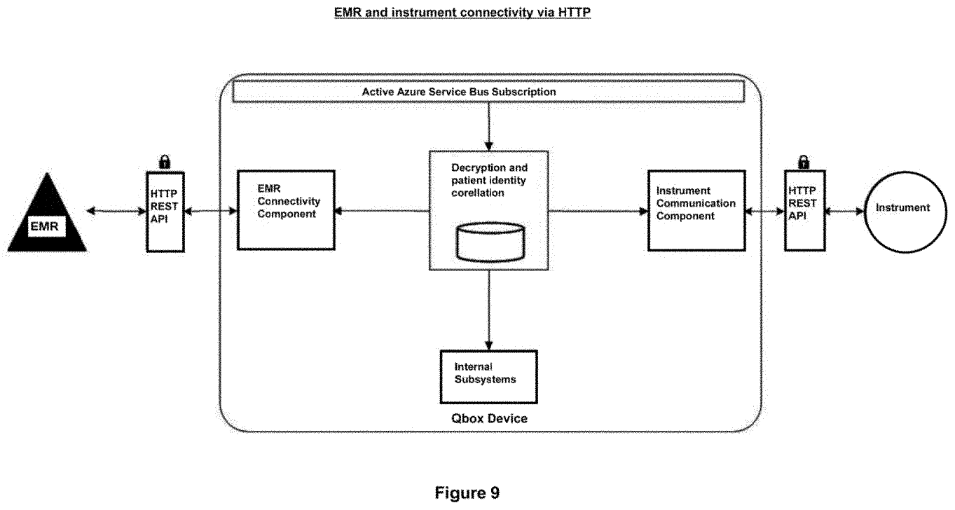

Using a myriad of expressive messages and events, the hub 1 can automatically synchronize devices (clinic 2 and home-based 4) with EMRs 3, helping to save time and reduce the potential for error in providing the medical or clinical service to a patient. The hub 1 can also connect with other external services such as network-based laboratory monitoring, data logging 6 and alarm systems. Remote monitoring is achieved by the hub 1 logging operational data to the cloud, whilst observing privacy standards. The level of detail being logged can be tailored to address various remote support scenarios. The hub 1 also facilitates secure external data transmission by employing a cloud-based message queue 7 operated in the cloud 8. This approach avoids the persistent storage of data in the cloud 8, because the message queue 7 is configured to store data only until sufficient time has passed for it to be delivered to the intended receiver. The message queue 7 can also be configured to include a time limit, for example, a time-to-live threshold, to further limit the amount of time data can persist on the queue 7. Once this time limit is reached, the data is deleted from the queue 7. In other words, upon the expiry of the transient period required for message delivery of individual packets or bundles of data, the messages in the queue will be deleted. Preferably, this time to live for queued messages is in the order of about 1 minute or less. In a preferred embodiment, patient data is stored locally in the hub 1. The hub 1 exchanges data securely with a mobile application 9, associated with a patient by residing on a mobile communication device 4 of the patient, through the cloud-based message queue 7. The message queue 7 of preferred embodiments is a standard First-In-First-Out queue and many services exist which can be used. The person skilled in the art will appreciate there are numerous options available for appropriate message queue functionality that may be adapted to provide a message queue for implementation in preferred embodiments of the invention. The preferred underlying technology for embodying the message queue 7 is AMQP (Advanced Message Queuing Protocol). AMQP 1.0 is a preferred international standard protocol for implementation. The connectivity between EMR's and instruments used by a patient at home is schematically illustrated in FIG. 9 using an HTTP based API as an example.

The hub 1 manages the flow of data to and from instruments and information systems by correlating all data moving in both directions. This correlation underpins the hub's ability to move data from a data source to the correct data recipient. The hub 1 is able to store partial data as it is received and later correlate it to form meaningful messages for connected systems once all the required data has been collected, and only then forward the complete data to the relevant data recipient in the format it expects.

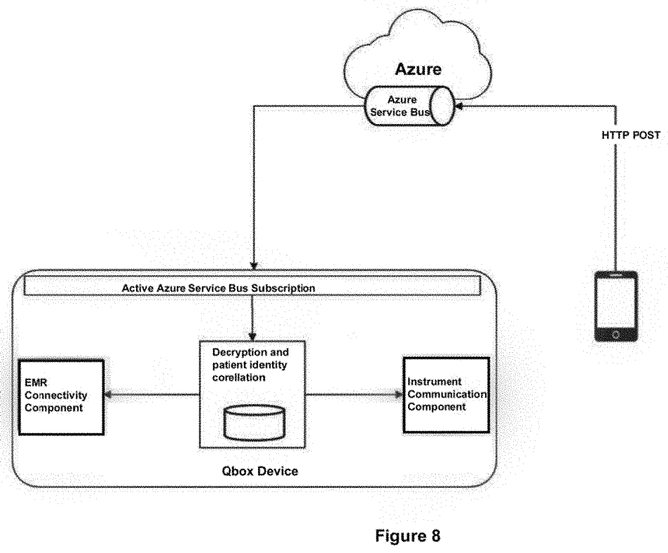

An example implementation of a cloud-based queue with transient communication and/or storage of data is schematically illustrated in FIG. 8 using an example Azure Service Bus. Alternatively, other proprietary message queue facilities may be utilised, such as for example, Amazon or Google cloud offerings. Equally, it is envisaged that a private cloud message system may be deployed to effect the appropriate communication of information in a message queue.

To facilitate this correlation and connectivity, the hub device has a local encrypted storage of identification and correlation information. This information consists of, but is not limited to.
a. Unique Patient identifiers: These identifiers are the unique identifiers generated by integrated clinic systems and instruments, and are used by the hub 1 to communicate patient data and events to and from these systems in an unambiguous fashion. Examples include, Patient Medical record number, procedure identifier (cycle number), patient system identifiers.
b. Patient identifiable information: These are additional identification fields that when combined can be used for patient identification, for instance patient name, telephone number or address.
c. Patient medical records: Due to the role of the hub 1 in correlating and transmitting events and information that pertains to the patient treatment in the clinic there are some instances where patient treatment information (including overall progress, procedures performed and outcomes) that are stored on the hub either temporarily to assist in correlation and transmission, or permanently to provide reporting capabilities. This could include the start and end dates of treatment, procedures performed during treatment, and the outcomes of tests performed by instruments both at home and in the clinic.
d. Linked Patient Identifiers: These are identifiers generated by the Hub and that can only be used to correlate back to, and identify the patient concerned using the other information stored on the Hub. There is no natural correlation between these identifiers and the patient.
e. Encryption and decryption keys: Any keys required to encrypt and decrypt messages communicated via the message queue discussed herein are stored on the hub, and not stored or transmitted via the message queue. A set of keys are also to be stored in the mobile application (established during the onboarding process, see below) so it can encrypt messages to the hub and decrypt messages received. These will be the alternate pairs of the keys in a standard public-private key exchange (each interaction with a mobile device involves a pair of keys, exchanged between the hub and the mobile application). The person skilled in the art will appreciate the operation and function of standard public-private key exchanges for secure cryptographic communication.

It is envisaged that more than one hub 1 may be provided at one or more clinics. Each hub 1 may have a dedicated message queue 7 and only authenticated users could access that particular dedicated queue 7. In use, preferred embodiments of the invention may utilise cloud-based message queue services such as but not limited to the Azure™ Service Bus Queue and the Amazon™ Simple Queue Service, which provide first-in first-out (FIFO) message queuing and allow a time-to-live to be set for each message. Message queues may provide a number of other advantages, including performance, reliability and scalability.

A linked patient identifier is preferably used as a substitute for direct patient identifiers when data is transmitted. For example, an identifier could be generated in the hub 1, such as, “3f506fe8-679d-49fd-8bed-42c0fa8fcff5”, for instance, which is in no way related to any patient or personal identifying information, as it is completely random. By way of explanation, a direct patient identifier is information that can be used alone to identify a patient, such as the patient's name or medical record number. Before transmission through the message queue 7, patient data is partially de-identified by removing direct patient identifiers. The partially de-identified patient data is then encrypted and a linked patient identifier is added. Linked patient identifiers may also be removed before transmission. The linked patient identifier is unique to the patient and is the only unencrypted data element in the system. The linked patient identifier cannot be used by other parties to identify the patient. The hub 1 is the only device with access to information that could identify the patient from the linked patient identifier.

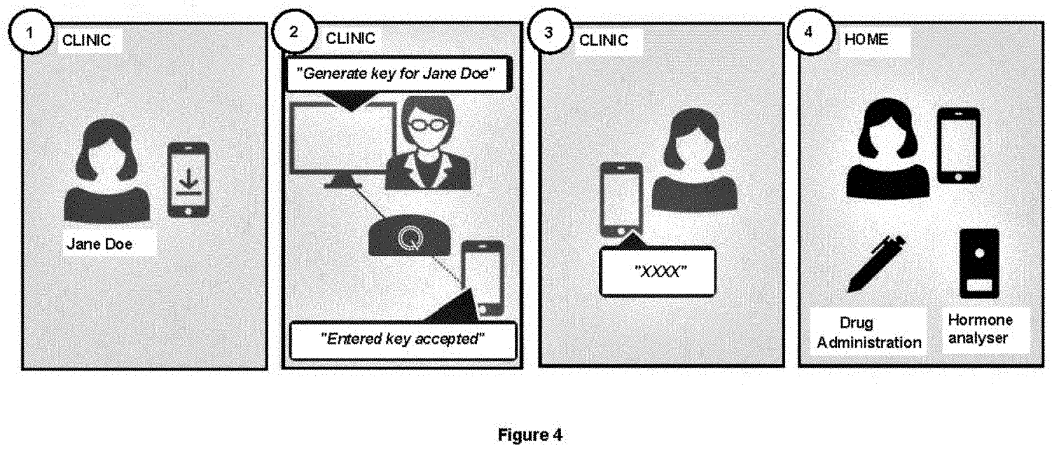

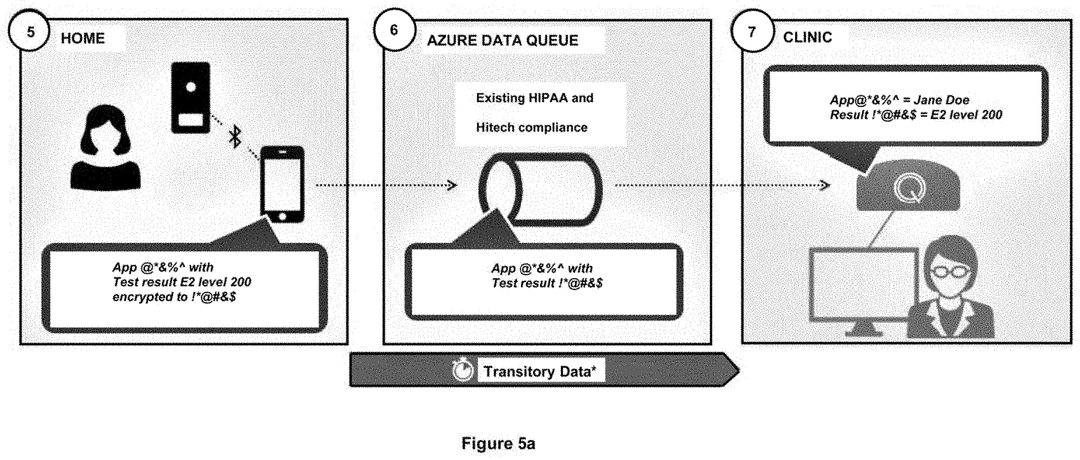

An onboarding process to familiarise a user such as a patient with their own use of medical products, which will be operated as linked devices 11 and the communication of data produced by those linked devices 11 is provided which links the mobile application 9 uniquely to the patient, and the mobile application 9 can then employ the linked patient identifier, cryptographic keys and the cloud-based message queue 7 to securely communicate with the hub 1. This enables secure data transmission between the mobile application 9 and the patient's EMRs 3, for example. At least some of the onboarding process could be facilitated by the scanning of a machine-readable code (such as a QR code or other barcode generated by the hub) by the mobile application 9 on the patient's personal device. An example onboarding process is shown in FIG. 4. Onboarding a patient is the process of registering the patient's mobile application with the hub 1 and linking the application to the identity of the patient in the clinic. The process includes the exchange of encryption keys and queue connectivity details.

Referring to FIG. 4, at step 1, a patient, Jane Doe, begins her fertility treatment cycle and will need to use home devices in this treatment. She downloads a device management App to her mobile device, eg her phone. At step 2 the clinic physician generates a unique ID to be embedded in the App which links it to Jane Doe's EMR file(s). As illustrated, upon generation of a key, it is entered and accepted by the App. At step 3, Jane then generates her own unique PIN or passcode to access the App. At step 4, after a briefing on how to use the devices, they can then be taken home by Jane for use in her treatment. Both the “key” and “ID” mentioned refer to the linked patient identifier. The hub 1 generates a unique identifier (the linked patient identifier) which links the patient app to the EMR record, and only the hub 1 can correlate that linked identifier back to the EMR record. When the patient app sends data to the clinic, the hub 1 uses the linked identifier to (1) find the private encryption key for that patient, (2) decrypts the data using that private key, (3) send the decrypted data to all interested systems and instruments.

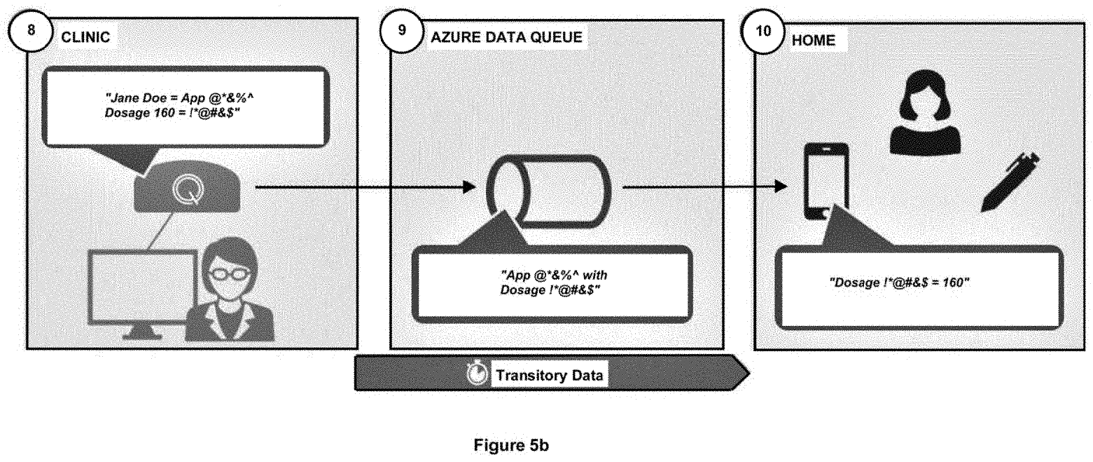

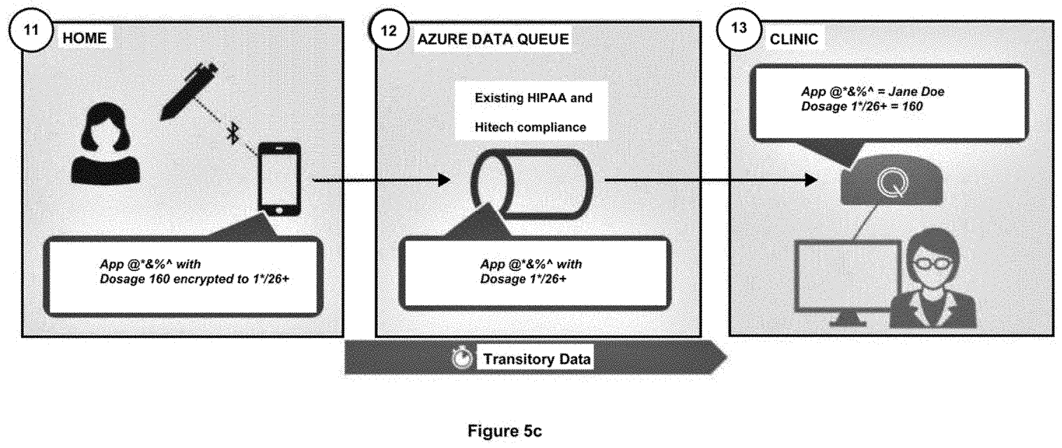

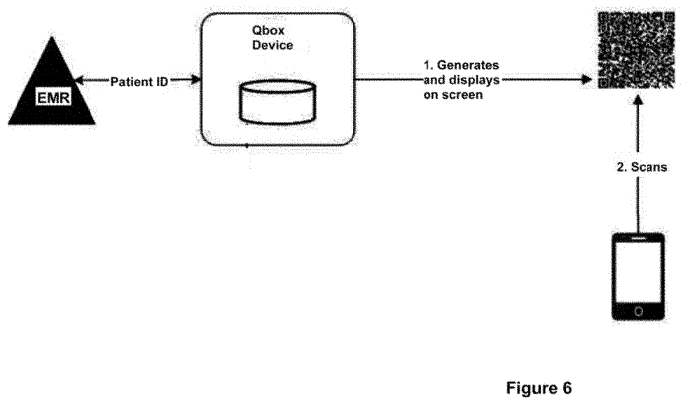

Referring to step 5 as shown in FIG. 5a, in use, for example at home, Jane, the patient, makes use of a device such as a hormone analyser. The results are first encrypted by the App and then sent to the data queue. As shown at step 6 the dedicated clinic data queue does not store data indefinitely, it resides there until it is requested by the clinic. At step 7 the hub 1 decrypts the data and then correlates the decrypted information back to the patient using the unique identifiers.

Steps 8, 9 and 10 are shown in FIG. 5b. At step 8 the physician updates a dosage for a specific patient based on hormone readings. The hub 1 encrypts the dosage information and sends it to the data queue. At step 9 the information passes transiently through the data queue. At step 10 the patient App receives the information and decodes it which can then be read by the patient for their use at home.

Steps 11, 12 and 13 are shown in FIG. 5c. At step 11 Jane uses a SmartCAP™ pen. The administered dosage is first encrypted and then sent to the data queue. At step 12 the information passes through the queue. At step 13 the hub 1 at the clinic decrypts the data then correlates the information back to the patient using the unique identifiers. With this, the physician can confirm compliance with the set medical protocol.

The patient is instructed by a healthcare clinic to install a mobile application on their personal device 4, for example, a smart phone. At the clinic, the mobile application 9 and hub 1 are able to communicate wirelessly. The hub 1 is operated to generate a linked patient identifier and cryptographic keys (e.g. public-key cryptography), which are communicated to the mobile application 9. The mobile application 9 is password protected, with a password created by the patient. Optionally, devices can then be paired with the mobile application 9. Another example embodiment of the onboarding process ensures that the machine-readable code (such as QR code) can only be used once. This is implemented by two-phase exchange of data between the mobile application and the hub in the clinic:
a) The machine-readable code is generated by the hub 1 and scanned by the patient's device 4. The code provides information for the mobile application 9 to connect to the message queue 7. Upon successful connection with the message queue 7, the mobile application 9 provides an acknowledgement to the hub 1. The hub ensures that the linked patient identifier can only be issued for onboarding once, in effect expiring the code and limiting its potential for misuse by others.
b) The hub 1, upon receiving the acknowledgement of successful connection from the mobile application 9, provides a response including the linked patient identifier, to provide enough information to enable the mobile application 9 to provide data (including partially de-identified and encrypted data) that can be understood and correlated to the patient (including EMR) by the hub 1.

In this specific instance, as pertains to the queue mechanism in a preferred embodiment of the invention, the mobile application 9 stores;

a. A public key for encrypting messages (the private key is secured on the hub)
b. A private key for decrypting messages (the public key is secured on the hub)
c. A linked patient identifier for identifying the sender/originator of messages
d. Messages and results (payload) that are yet to be communicated to the hub 1.
e. Messages and other notifications received from the hub 1 and not yet deleted by the user/patient.

With reference to FIG. 7, devices, such as blue-tooth or Wi-Fi enabled sensors for home use, can be paired to the mobile application 9 for monitoring at the patient's home 200 under a home monitoring procedure, including to facilitate feedback on dosages and adherence to protocols. When a result is taken from the home device, or some other communication needs to be sent to the clinic or physician, then this message is communicated to the mobile device (potentially via human interaction, Bluetooth, or another mechanism) and the device encrypts the payload using the encryption key and publishes a notification to the message queue 7, tagged with the linked patient identifier. This message is received by the Hub 1 via subscription provided as part of the AMQP specification for a FIFO queue. Essentially the hub 1 subscribes to the queue 7 so that any messages which are added to the queue 7 by the mobile device are automatically forwarded to the hub 1 as the subscriber. Internally, the hub 1 has multiple subsystems and components which communicate in a similar manner. In other words, the hub 1 itself has queuing technology which it uses to publish messages to interested subsystems. It takes the received message from the cloud queue, formats it into a new message which is easier to deal with internally, and then puts that new message onto a new queue which only software on the hub 1 has access to. All the subsystems subscribe to the internal queue, and in so doing, make it very simple for messages, or “notifications” to be sent to them in this manner.

If the message is expected (correct format, and linked user is registered) and the encryption key used is correct (was the key assigned to the user) then the result or payload is decrypted and the relevant systems notified of the contents. The hub 1 performs this notification using the internally correlated unique patient identifiers and any configured routing rules and mechanisms.

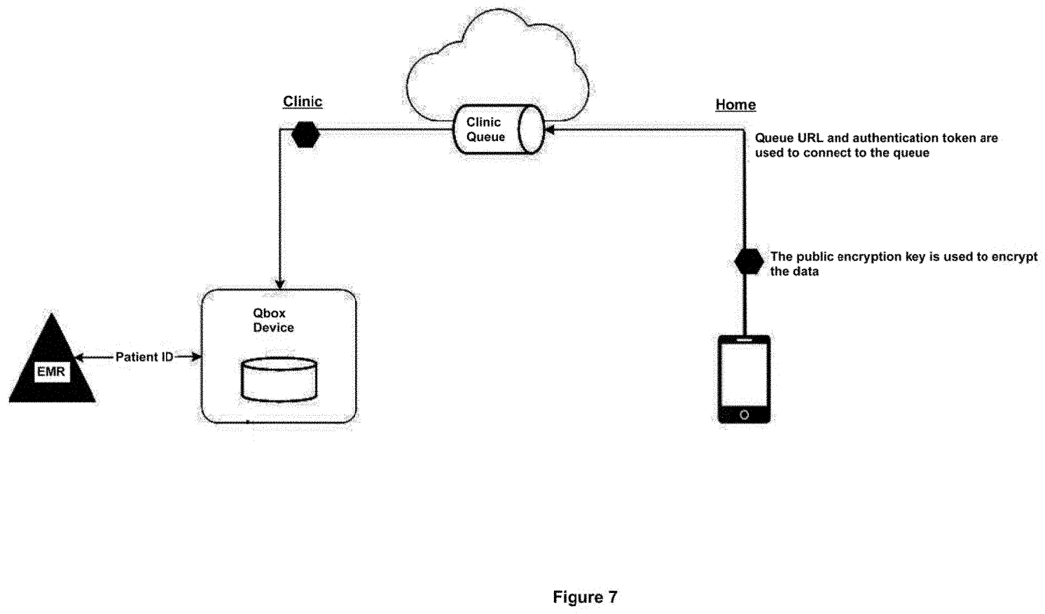

As noted above, FIGS. 5a, 5b and 5c illustrate an example application to home-based monitoring in accordance with preferred embodiments. In this example, the patient uses a linked device 11, such as a hormone analyzer device paired to the mobile application 9. The mobile application 9 transmits the reading with the linked patient identifier to the hub 1 via a message queue 7. The hub 1 decrypts the data at the clinic 100. The clinician may then update the dose, and this information is transmitted by the hub 1 to the mobile application 9 via message queue 7. The patient receives the updated dose on the mobile application 9. The patient administers the dose using a paired device 11. The mobile application 9 transmits the dosage administered and the linked patient identifier to the hub 1 via a message queue 7. At various points in time, the hub 1 is able to update the patient's EMRs 3. At least some of the device pairing process may be facilitated by the scanning of a readable code (such as a QR code or other barcode generated by the hub 1), with suitable functionality of the mobile application 9 able to be unlocked once paired with the device. For example, before pairing, the mobile application might only function as a standalone application, and features may include providing basic information such as guidance and education. Once the mobile application 9 is paired and a linked patient identifier is established, the application could make available a suite of device and/or clinic-specific functionality by exposing new features dynamically. These could include patient schedules and alerts and updates from the clinic. FIG. 6 shows the hub 1 or Qbox device with a database containing linked patient identifier correlated to EMR patient identifier. A QR code is generated and displayed on screen. This will include the patient linked identifier as a security key, a public encryption key, a queue URL and a queue authentication token as an author token. The mobile application will now have all information required to encrypt data, connect to the clinic queue and place encrypted data on the clinic queue. Additionally, once the QR code is scanned, advanced features of the App may be unlocked for a user, such as patient schedules. It is important to note that the App may be available to any person via an App store, so it at least has some basic functionality, however, in order to benefit from the enriched functionality, the user must be a patient of the clinic with a hub 1.

The solution of preferred embodiments described above has been described in the context of a clinic with patients. Alternatively, the solution could be deployed in other contexts involving sensitive data. For example, pathology clinics where connectivity to Lab Information Systems (LIS) is required, and hospitals where access to Hospital Information Systems and other EMRs is required.

Some data transmission between the mobile application and the hub could occur outside of the cloud-based message queue, for example for transmission of less sensitive data. Options for transmission of less sensitive data include the use of encrypted email, SSH file transfer protocol, or a cloud-based database to which all parties have access.

The use of cloud-based message queues is not necessarily limited to data transmission between the hub and patients. The hub could communicate with other external entities, such as other healthcare clinics, with each clinic employing a local hub and a cloud-based message queue. This could for example facilitate providers running multiple clinics and/or patients moving between clinics.

While this invention has been described in connection with specific embodiments thereof, it will be understood that it is capable of further modification(s). This application is intended to cover any variations uses or adaptations of the invention following in general, the principles of the invention and including such departures from the present disclosure as come within known or customary practice within the art to which the invention pertains and as may be applied to the essential features hereinbefore set forth.

As the present invention may be embodied in several forms without departing from the spirit of the essential characteristics of the invention, it should be understood that the above described embodiments are not to limit the present invention unless otherwise specified, but rather should be construed broadly within the spirit and scope of the invention as defined in the appended claims. The described embodiments are to be considered in all respects as illustrative only and not restrictive.

Various modifications and equivalent arrangements are intended to be included within the spirit and scope of the invention and appended claims. Therefore, the specific embodiments are to be understood to be illustrative of the many ways in which the principles of the present invention may be practiced. In the following claims, any means-plus-function clauses are intended to cover structures as performing the defined function and not only structural equivalents, but also equivalent structures. For example, although a nail and a screw may not be structural equivalents in that a nail employs a cylindrical surface to secure wooden parts together, whereas a screw employs a helical surface to secure wooden parts together, in the environment of fastening wooden parts, a nail and a screw are equivalent structures.

The following sections I-VII provide a guide to interpreting the present specification.

### I. Terms

The term “mobile application program” is to be understood to be reference to a complete, self-contained computer-processor-implemented program that performs a specific function directly for a user. This is in contrast to system software such as the operating system kernel, server processes, libraries which exists to support application programs and utility programs. The term is also to be taken as synonymous with the terms “App”, “app”, and “mobile application”.

The term “product” means any machine, manufacture and/or composition of matter, unless expressly specified otherwise.

The term “process” means any process, algorithm, method or the like, unless expressly specified otherwise.

Each process (whether called a method, algorithm or otherwise) inherently includes one or more steps, and therefore all references to a “step” or “steps” of a process have an inherent antecedent basis in the mere recitation of the term ‘process’ or a like term. Accordingly, any reference in a claim to a ‘step’ or ‘steps’ of a process has sufficient antecedent basis.

The term “invention” and the like mean “the one or more inventions disclosed in this specification”, unless expressly specified otherwise.

The terms “an embodiment”, “embodiment”, “embodiments”, “the embodiment”, “the embodiments”, “one or more embodiments”, “some embodiments”, “certain embodiments”, “one embodiment”, “another embodiment” and the like mean “one or more (but not all) embodiments of the disclosed invention(s)”, unless expressly specified otherwise.

The term “variation” of an invention means an embodiment of the invention, unless expressly specified otherwise.

A reference to “another embodiment” in describing an embodiment does not imply that the referenced embodiment is mutually exclusive with another embodiment (e.g., an embodiment described before the referenced embodiment), unless expressly specified otherwise.

The terms “including”, “comprising” and variations thereof mean “including but not limited to”, unless expressly specified otherwise.

The terms “a”, “an” and “the” mean “one or more”, unless expressly specified otherwise.

The term “plurality” means “two or more”, unless expressly specified otherwise.

The term “herein” means “in the present specification, including anything which may be incorporated by reference”, unless expressly specified otherwise.

The phrase “at least one of”, when such phrase modifies a plurality of things (such as an enumerated list of things), means any combination of one or more of those things, unless expressly specified otherwise. For example, the phrase “at least one of a widget, a car and a wheel” means either (i) a widget, (ii) a car, (iii) a wheel, (iv) a widget and a car, (v) a widget and a wheel, (vi) a car and a wheel, or (vii) a widget, a car and a wheel. The phrase “at least one of”, when such phrase modifies a plurality of things, does not mean “one of each of” the plurality of things.

Numerical terms such as “one”, “two”, etc. when used as cardinal numbers to indicate quantity of something (e.g., one widget, two widgets), mean the quantity indicated by that numerical term, but do not mean at least the quantity indicated by that numerical term. For example, the phrase “one widget” does not mean “at least one widget”, and therefore the phrase “one widget” does not cover, e.g., two widgets.

The phrase “based on” does not mean “based only on”, unless expressly specified otherwise. In other words, the phrase “based on” describes both “based only on” and “based at least on”. The phrase “based at least on” is equivalent to the phrase “based at least in part on”.

The term “represent” and like terms are not exclusive, unless expressly specified otherwise. For example, the term “represents” do not mean “represents only”, unless expressly specified otherwise. In other words, the phrase “the data represents a credit card number” describes both “the data represents only a credit card number” and “the data represents a credit card number and the data also represents something else”.

The term “whereby” is used herein only to precede a clause or other set of words that express only the intended result, objective or consequence of something that is previously and explicitly recited. Thus, when the term “whereby” is used in a claim, the clause or other words that the term “whereby” modifies do not establish specific further limitations of the claim or otherwise restricts the meaning or scope of the claim.

The term “e.g.” and like terms mean “for example”, and thus does not limit the term or phrase it explains. For example, in the sentence “the computer sends data (e.g., instructions, a data structure) over the Internet”, the term “e.g.” explains that “instructions” are an example of “data” that the computer may send over the Internet, and also explains that “a data structure” is an example of “data” that the computer may send over the Internet. However, both “instructions” and “a data structure” are merely examples of “data”, and other things besides “instructions” and “a data structure” can be “data”.

The term “i.e.” and like terms mean “that is”, and thus limits the term or phrase it explains. For example, in the sentence “the computer sends data (i.e., instructions) over the Internet”, the term “i.e.” explains that “instructions” are the “data” that the computer sends over the Internet.

Any given numerical range shall include whole and fractions of numbers within the range. For example, the range “1 to 10” shall be interpreted to specifically include whole numbers between 1 and 10 (e.g., 2, 3, 4, . . . 9) and non-whole numbers (e.g., 1.1, 1.2, . . . 1.9).

### II. Determining

The term “determining” and grammatical variants thereof (e.g., to determine a price, determining a value, determine an object which meets a certain criterion) is used in an extremely broad sense. The term “determining” encompasses a wide variety of actions and therefore “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing, and the like.

The term “determining” does not imply certainty or absolute precision, and therefore “determining” can include estimating, extrapolating, predicting, guessing and the like.

The term “determining” does not imply that mathematical processing must be performed, and does not imply that numerical methods must be used, and does not imply that an algorithm or process is used.

The term “determining” does not imply that any particular device must be used. For example, a computer need not necessarily perform the determining.

### III. Indication

The term “indication” is used in an extremely broad sense. The term “indication” may, among other things, encompass a sign, symptom, or token of something else.

The term “indication” may be used to refer to any indicia and/or other information indicative of or associated with a subject, item, entity, and/or other object and/or idea.

As used herein, the phrases “information indicative of” and “indicia” may be used to refer to any information that represents, describes, and/or is otherwise associated with a related entity, subject, or object.

Indicia of information may include, for example, a symbol, a code, a reference, a link, a signal, an identifier, and/or any combination thereof and/or any other informative representation associated with the information.

In some embodiments, indicia of information (or indicative of the information) may be or include the information itself and/or any portion or component of the information. In some embodiments, an indication may include a request, a solicitation, a broadcast, and/or any other form of information gathering and/or dissemination.

### IV. Forms of Sentences

Where a limitation of a first claim would cover one of a feature as well as more than one of a feature (e.g., a limitation such as “at least one widget” covers one widget as well as more than one widget), and where in a second claim that depends on the first claim, the second claim uses a definite article “the” to refer to the limitation (e.g., “the widget”), this does not imply that the first claim covers only one of the feature, and this does not imply that the second claim covers only one of the feature (e.g., “the widget” can cover both one widget and more than one widget).

When an ordinal number (such as “first”, “second”, “third” and so on) is used as an adjective before a term, that ordinal number is used (unless expressly specified otherwise) merely to indicate a particular feature, such as to distinguish that particular feature from another feature that is described by the same term or by a similar term. For example, a “first widget” may be so named merely to distinguish it from, e.g., a “second widget”. Thus, the mere usage of the ordinal numbers “first” and “second” before the term “widget” does not indicate any other relationship between the two widgets, and likewise does not indicate any other characteristics of either or both widgets. For example, the mere usage of the ordinal numbers “first” and “second” before the term “widget” (1) does not indicate that either widget comes before or after any other in order or location; (2) does not indicate that either widget occurs or acts before or after any other in time; and (3) does not indicate that either widget ranks above or below any other, as in importance or quality. In addition, the mere usage of ordinal numbers does not define a numerical limit to the features identified with the ordinal numbers. For example, the mere usage of the ordinal numbers “first” and “second” before the term “widget” does not indicate that there must be no more than two widgets.

When a single device or article is described herein, more than one device/article (whether or not they cooperate) may alternatively be used in place of the single device/article that is described. Accordingly, the functionality that is described as being possessed by a device may alternatively be possessed by more than one device/article (whether or not they cooperate).

Similarly, where more than one device or article is described herein (whether or not they cooperate), a single device/article may alternatively be used in place of the more than one device or article that is described. For example, a plurality of computer-based devices may be substituted with a single computer-based device. Accordingly, the various functionality that is described as being possessed by more than one device or article may alternatively be possessed by a single device/article.

The functionality and/or the features of a single device that is described may be alternatively embodied by one or more other devices which are described but are not explicitly described as having such functionality/features. Thus, other embodiments need not include the described device itself, but rather can include the one or more other devices which would, in those other embodiments, have such functionality/features.

### V. Disclosed Examples and Terminology are not Limiting

Neither the Title nor the Abstract in this specification is intended to be taken as limiting in any way as the scope of the disclosed invention(s). The title and headings of sections provided in the specification are for convenience only, and are not to be taken as limiting the disclosure in any way.

Numerous embodiments are described in the present application, and are presented for illustrative purposes only. The described embodiments are not, and are not intended to be, limiting in any sense. The presently disclosed invention(s) are widely applicable to numerous embodiments, as is readily apparent from the disclosure. One of ordinary skill in the art will recognise that the disclosed invention(s) may be practised with various modifications and alterations, such as structural, logical, software, and electrical modifications. Although particular features of the disclosed invention(s) may be described with reference to one or more particular embodiments and/or drawings, it should be understood that such features are not limited to usage in the one or more particular embodiments or drawings with reference to which they are described, unless expressly specified otherwise.

The present disclosure is not a literal description of all embodiments of the invention(s). Also, the present disclosure is not a listing of features of the invention(s) which must be present in all embodiments.

Devices that are described as in communication with each other need not be in continuous communication with each other, unless expressly specified otherwise. On the contrary, such devices need only transmit to each other as necessary or desirable, and may actually refrain from exchanging data most of the time. For example, a machine in communication with another machine via the Internet may not transmit data to the other machine for long period of time (e.g. weeks at a time). In addition, devices that are in communication with each other may communicate directly or indirectly through one or more intermediaries.

A description of an embodiment with several components or features does not imply that all or even any of such components/features are required. On the contrary, a variety of optional components are described to illustrate the wide variety of possible embodiments of the present invention(s). Unless otherwise specified explicitly, no component/feature is essential or required.

Although process steps, operations, algorithms or the like may be described in a particular sequential order, such processes may be configured to work in different orders. In other words, any sequence or order of steps that may be explicitly described does not necessarily indicate a requirement that the steps be performed in that order. The steps of processes described herein may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to the invention(s), and does not imply that the illustrated process is preferred.

Although a process may be described as including a plurality of steps, that does not imply that all or any of the steps are preferred, essential or required. Various other embodiments within the scope of the described invention(s) include other processes that omit some or all of the described steps. Unless otherwise specified explicitly, no step is essential or required.

Although a process may be described singly or without reference to other products or methods, in an embodiment the process may interact with other products or methods. For example, such interaction may include linking one business model to another business model. Such interaction may be provided to enhance the flexibility or desirability of the process.

Although a product may be described as including a plurality of components, aspects, qualities, characteristics and/or features, that does not indicate that any or all of the plurality are preferred, essential or required. Various other embodiments within the scope of the described invention(s) include other products that omit some or all of the described plurality.

An enumerated list of items (which may or may not be numbered) does not imply that any or all of the items are mutually exclusive, unless expressly specified otherwise. Likewise, an enumerated list of items (which may or may not be numbered) does not imply that any or all of the items are comprehensive of any category, unless expressly specified otherwise. For example, the enumerated list “a computer, a laptop, a PDA” does not imply that any or all of the three items of that list are mutually exclusive and does not imply that any or all of the three items of that list are comprehensive of any category.

An enumerated list of items (which may or may not be numbered) does not imply that any or all of the items are equivalent to each other or readily substituted for each other.

All embodiments are illustrative, and do not imply that the invention or any embodiments were made or performed, as the case may be.

### VI. Computing

It will be readily apparent to one of ordinary skill in the art that the various processes described herein may be implemented by, e.g., appropriately programmed general purpose computers, special purpose computers and computing devices. Typically a processor (e.g., one or more microprocessors, one or more micro-controllers, one or more digital signal processors) will receive instructions (e.g., from a memory or like device), and execute those instructions, thereby performing one or more processes defined by those instructions.

A “processor” means one or more microprocessors, central processing units (CPUs), computing devices, micro-controllers, digital signal processors, or like devices or any combination thereof.

Thus a description of a process is likewise a description of an apparatus for performing the process. The apparatus that performs the process can include, e.g., a processor and those input devices and output devices that are appropriate to perform the process.

Further, programs that implement such methods (as well as other types of data) may be stored and transmitted using a variety of media (e.g., computer readable media) in a number of manners. In some embodiments, hard-wired circuitry or custom hardware may be used in place of, or in combination with, some or all of the software instructions that can implement the processes of various embodiments. Thus, various combinations of hardware and software may be used instead of software only.

The term “computer-readable medium” refers to any medium, a plurality of the same, or a combination of different media, that participate in providing data (e.g., instructions, data structures) which may be read by a computer, a processor or a like device. Such a medium may take many forms, including but not limited to, non-volatile media, volatile media, and transmission media. Non-volatile media include, for example, optical or magnetic disks and other persistent memory. Volatile media include dynamic random access memory (DRAM), which typically constitutes the main memory. Transmission media include coaxial cables, copper wire and fibre optics, including the wires that comprise a system bus coupled to the processor. Transmission media may include or convey acoustic waves, light waves and electromagnetic emissions, such as those generated during radio frequency (RF) and infra-red (IR) data communications. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM, a FLASH-EEPROM, any other memory chip or cartridge, a carrier wave as described hereinafter, or any other medium from which a computer can read.

Various forms of computer readable media may be involved in carrying data (e.g. sequences of instructions) to a processor. For example, data may be (i) delivered from RAM to a processor; (ii) carried over a wireless transmission medium; (iii) formatted and/or transmitted according to numerous formats, standards or protocols, such as Ethernet (or IEEE 802.3), SAP, ATP, Bluetooth™, and TCP/IP, TDMA, CDMA, and 3G; and/or (iv) encrypted to ensure privacy or prevent fraud in any of a variety of ways well known in the art.

Thus a description of a process is likewise a description of a computer-readable medium storing a program for performing the process. The computer-readable medium can store (in any appropriate format) those program elements which are appropriate to perform the method.

Just as the description of various steps in a process does not indicate that all the described steps are required, embodiments of an apparatus include a computer/computing device operable to perform some (but not necessarily all) of the described process.

Likewise, just as the description of various steps in a process does not indicate that all the described steps are required, embodiments of a computer-readable medium storing a program or data structure include a computer-readable medium storing a program that, when executed, can cause a processor to perform some (but not necessarily all) of the described process.

Where databases are described, it will be understood by one of ordinary skill in the art that (i) alternative database structures to those described may be readily employed, and (ii) other memory structures besides databases may be readily employed. Any illustrations or descriptions of any sample databases presented herein are illustrative arrangements for stored representations of information. Any number of other arrangements may be employed besides those suggested by, e.g., tables illustrated in drawings or elsewhere. Similarly, any illustrated entries of the databases represent exemplary information only; one of ordinary skill in the art will understand that the number and content of the entries can be different from those described herein. Further, despite any depiction of the databases as tables, other formats (including relational databases, object-based models and/or distributed databases) could be used to store and manipulate the data types described herein. Likewise, object methods or behaviours of a database can be used to implement various processes, such as the described herein. In addition, the databases may, in a known manner, be stored locally or remotely from a device which accesses data in such a database.

Various embodiments can be configured to work in a network environment including a computer that is in communication (e.g., via a communications network) with one or more devices. The computer may communicate with the devices directly or indirectly, via any wired or wireless medium (e.g. the Internet, LAN, WAN or Ethernet, Token Ring, a telephone line, a cable line, a radio channel, an optical communications line, commercial on-line service providers, bulletin board systems, a satellite communications link, a combination of any of the above). Each of the devices may themselves comprise computers or other computing devices that are adapted to communicate with the computer. Any number and type of devices may be in communication with the computer.

In an embodiment, a server computer or centralised authority may not be necessary or desirable. For example, the present invention may, in an embodiment, be practised on one or more devices without a central authority. In such an embodiment, any functions described herein as performed by the server computer or data described as stored on the server computer may instead be performed by or stored on one or more such devices.

Where a process is described, in an embodiment the process may operate without any user intervention. In another embodiment, the process includes some human intervention (e.g., a step is performed by or with the assistance of a human).

It should be noted that where the terms “server”, “secure server” or similar terms are used herein, a communication device is described that may be used in a communication system, unless the context otherwise requires, and should not be construed to limit the present invention to any particular communication device type. Thus, a communication device may include, without limitation, a bridge, router, bridge-router (router), switch, node, or other communication device, which may or may not be secure.

It should also be noted that where a flowchart is used herein to demonstrate various aspects of the invention, it should not be construed to limit the present invention to any particular logic flow or logic implementation. The described logic may be partitioned into different logic blocks (e.g., programs, modules, functions, or subroutines) without changing the overall results or otherwise departing from the true scope of the invention. Often, logic elements may be added, modified, omitted, performed in a different order, or implemented using different logic constructs (e.g., logic gates, looping primitives, conditional logic, and other logic constructs) without changing the overall results or otherwise departing from the true scope of the invention.

Various embodiments of the invention may be embodied in many different forms, including computer program logic for use with a processor (e.g., a microprocessor, microcontroller, digital signal processor, or general purpose computer and for that matter, any commercial processor may be used to implement the embodiments of the invention either as a single processor, serial or parallel set of processors in the system and, as such, examples of commercial processors include, but are not limited to Merced™, Pentium™, Pentium II™, Xeon™, Celeron™, Pentium Pro™, Efficeon™, Athlon™, AMD™ and the like), programmable logic for use with a programmable logic device (e.g., a Field Programmable Gate Array (FPGA) or other PLD), discrete components, integrated circuitry (e.g., an Application Specific Integrated Circuit (ASIC)), or any other means including any combination thereof. In an exemplary embodiment of the present invention, predominantly all of the communication between users and the server is implemented as a set of computer program instructions that is converted into a computer executable form, stored as such in a computer readable medium, and executed by a microprocessor under the control of an operating system.

Computer program logic implementing all or part of the functionality where described herein may be embodied in various forms, including a source code form, a computer executable form, and various intermediate forms (e.g., forms generated by an assembler, compiler, linker, or locator). Source code may include a series of computer program instructions implemented in any of various programming languages (e.g., an object code, an assembly language, or a high-level language such as Fortran, C, C++, JAVA, or HTML. Moreover, there are hundreds of available computer languages that may be used to implement embodiments of the invention, among the more common being Ada; Algol; APL; awk; Basic; C; C++; Conol; Delphi; Eiffel; Euphoria; Forth; Fortran; HTML; Icon; Java; Javascript; Lisp; Logo; Mathematica; MatLab; Miranda; Modula-2; Oberon; Pascal; Perl; PL/I; Prolog; Python; Rexx; SAS; Scheme; sed; Simula; Smalltalk; Snobol; SQL; Visual Basic; Visual C++; Linux and XML.) for use with various operating systems or operating environments. The source code may define and use various data structures and communication messages. The source code may be in a computer executable form (e.g., via an interpreter), or the source code may be converted (e.g., via a translator, assembler, or compiler) into a computer executable form.

The computer program may be fixed in any form (e.g., source code form, computer executable form, or an intermediate form) either permanently or transitorily in a tangible storage medium, such as a semiconductor memory device (e.g, a RAM, ROM, PROM, EEPROM, or Flash-Programmable RAM), a magnetic memory device (e.g., a diskette or fixed disk), an optical memory device (e.g., a CD-ROM or DVD-ROM), a PC card (e.g., PCMCIA card), or other memory device. The computer program may be fixed in any form in a signal that is transmittable to a computer using any of various communication technologies, including, but in no way limited to, analog technologies, digital technologies, optical technologies, wireless technologies (e.g., Bluetooth), networking technologies, and inter-networking technologies. The computer program may be distributed in any form as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web).

Hardware logic (including programmable logic for use with a programmable logic device) implementing all or part of the functionality where described herein may be designed using traditional manual methods, or may be designed, captured, simulated, or documented electronically using various tools, such as Computer Aided Design (CAD), a hardware description language (e.g., VHDL or AHDL), or a PLD programming language (e.g., PALASM, ABEL, or CUPL). Hardware logic may also be incorporated into display screens for implementing embodiments of the invention and which may be segmented display screens, analogue display screens, digital display screens, CRTs, LED screens, Plasma screens, liquid crystal diode screen, and the like.

Programmable logic may be fixed either permanently or transitorily in a tangible storage medium, such as a semiconductor memory device (e.g., a RAM, ROM, PROM, EEPROM, or Flash-Programmable RAM), a magnetic memory device (e.g., a diskette or fixed disk), an optical memory device (e.g., a CD-ROM or DVD-ROM), or other memory device. The programmable logic may be fixed in a signal that is transmittable to a computer using any of various communication technologies, including, but in no way limited to, analog technologies, digital technologies, optical technologies, wireless technologies (e.g., Bluetooth), networking technologies, and internetworking technologies. The programmable logic may be distributed as a removable storage medium with accompanying printed or electronic documentation (e.g., shrink wrapped software), preloaded with a computer system (e.g., on system ROM or fixed disk), or distributed from a server or electronic bulletin board over the communication system (e.g., the Internet or World Wide Web).

“Comprises/comprising” and “includes/including” when used in this specification is taken to specify the presence of stated features, integers, steps or components but does not preclude the presence or addition of one or more other features, integers, steps, components or groups thereof. Thus, unless the context clearly requires otherwise, throughout the description and the claims, the words ‘comprise’, ‘comprising’, ‘includes’, ‘including’ and the like are to be construed in an inclusive sense as opposed to an exclusive or exhaustive sense; that is to say, in the sense of “including, but not limited to”.

1-30. (canceled)

31. A method of communicating patient data over an interconnected computer data network between a patient having a patient mobile communication device and a clinic having a local data management hub which is operatively connected to the interconnected computer data network and comprises processor means adapted to operate in accordance with a predetermined instruction set, the method comprising the steps of:
loading instructions from the predetermined instruction set for the encryption and decryption of patient data onto the patient mobile communication device for secure data communication where the patient data to be encrypted for including in the secure data communication is exclusive of direct patient identifiers;
loading instructions from the predetermined instruction set onto the patient mobile communication device for connecting to a transient data store which resides within the interconnected computer data network intermediate the patient mobile communication device and the clinic;
providing a secure communication of data that includes the encrypted patient data between the patient mobile communication device and the clinic wherein both the patient mobile communication device and the local data management hub of the clinic only transmit or receive data by subscription with the transient data store.

32. A method as claimed in claim 31 wherein the secure communication of data between the patient mobile communication device and the clinic further includes at least one linked patient identifier which, in combination with information stored only on the local data management hub, identifies a patient.

33. A method as claimed in claim 31 wherein the steps of loading instructions from the predetermined instruction set onto the patient mobile communication device are performed simultaneously by the patient mobile communication device scanning a machine-readable optical label that contains information comprising the respective instructions to be loaded.

34. A method as claimed in claim 31 wherein the patient data is stored only on one or a combination of either the local data management hub or the patient mobile device and wherein patient data is derived from instruments in communication with the patient mobile communication device.

35. A method as claimed in claim 31 further including the step of:
linking patient data received at the local data management hub from the patient mobile communication device with a patient's EMR.

36. A method as claimed in claim 31 wherein the instructions from the predetermined instruction set for the encryption and decryption of data include an encryption key unique for the patient.

37. A method as claimed in claim 31 wherein the transient data store is dedicated to the clinic and/or a message queue.

38. A method as claimed in claim 37 where the message queue has a defined time to live for queued messages of about 1 minute or less.

39. A method as claimed in claim 31 wherein the interconnected computer data network comprises one or a combination of:
an intranet;
a local area network;
a campus network;
a wide area network;
the internet.

40. A system for communicating patient data over an interconnected computer data network between a patient and a clinic, the system comprising:
a patient mobile communication device operably associated with the patient;
a local data management hub operable within the clinic and which is operatively connected to the interconnected computer data network and comprises processor means adapted to operate in accordance with a predetermined instruction set;
a transient data store which resides within the interconnected computer data network intermediate the patient mobile communication device and the clinic;
an application program adapted for being downloaded from the predetermined instruction set and residing on the patient mobile communication device and further adapted to;
load instructions from the predetermined instruction set onto the patient mobile communication device for the encryption and decryption of patient data for secure data communication where the patient data to be encrypted for including in the secure data communication is exclusive of direct patient identifiers; and
load instructions from the predetermined instruction set onto the patient mobile communication device for connecting to the transient data store;
wherein both the patient mobile communication device and the local data management hub of the clinic only transmit or receive data, that includes the encrypted patient data securely communicated between the patient mobile communication device and the clinic, by subscription with the transient data store.

41. A system as claimed in claim 40 wherein the securely communicated data between the patient mobile communication device and the clinic further includes at least one linked patient identifier which, in combination with information stored only on the local data management hub, identifies a patient.

42. A system as claimed in claim 40 wherein the local data management hub and the patient mobile communication device include storage means respectively for storing patient data.

43. A system as claimed in claim 40 further comprising medical instruments in communication with the patient mobile communication device for providing measurements from which the patient data is derived.

44. A system as claimed in claim 40 further including at least one patient EMR.

45. A system as claimed in claim 44 wherein the processor means of the local data management hub operating in accordance with the predetermined instruction set is adapted to link patient data received at the local data management hub from the patient mobile communication device with a patient's EMR.

46. A system as claimed in claim 40 wherein the instructions from the predetermined instruction set for the encryption and decryption of data include an encryption key unique for the patient.

47. A system as claimed in claim 40 wherein the transient data store is dedicated to the clinic and/or a message queue.

48. A system as claimed in claim 47 where the message queue has a defined time to live for queued messages of about 1 minute or less.

49. A system as claimed in claim 40 wherein the interconnected computer data network comprises one or a combination of:
an intranet;
a local area network;
a campus network;
a wide area network;
the internet.

50. Apparatus adapted to communicate patient data over an interconnected computer data network between a patient and a clinic, said apparatus comprising:
processor means adapted to operate in accordance with a predetermined instruction set,
said apparatus, in conjunction with said instruction set, being adapted to perform the method steps as claimed in claim 31.

51. A computer program product comprising:
a computer usable medium having computer readable program code and computer readable system code embodied on said medium for communicating patient data between a patient and a clinic within a data processing system over an interconnected computer data network, said computer program product comprising:
computer readable code within said computer usable medium for performing the method steps as claimed in claim 31.

52. A method of uniquely associating a patient with a clinical record that is communicable to a clinic, the patient having a patient mobile communication device and the clinic having a local data management hub that comprises processor means adapted to operate in accordance with a predetermined instruction set and which is operatively connected to an interconnected computer data network, the method comprising the steps of:
downloading an application program from the local data management hub onto the patient mobile communication device;
generating, at the clinic, a unique ID that links only the unique ID to an EMR of the patient;
embedding the unique ID into the downloaded application program;
creating a unique PIN for the patient to access the application program;
operatively associating one or more medical instruments for use by the patient with the application program;
communicating patient data created by the patient's use of the one or more medical instruments to the application program where loaded instructions from the predetermined instruction set onto the mobile communication device encrypt the patient data for secure data communication over the interconnected computer data network via a transient data store to the clinic;
decrypting the patient data at the local data management hub;
using the unique ID to correlate the patient data with the patient at the local data management hub.
